(19) United States
(12) Patent Application Publication
(10) Pub. No.: US 2005/0003509 A1
(43) Pub. Date: Jan. 6, 2005

(54) Method for modulating C.albicans

(76) Inventors: Per O. Ljungdahl, Stockholm (SE); Paula Martinez, Stockholm (SE)

Correspondence Address: FULBRIGHT & JAWORSKI, LLP, 666 FIFTH AVE, NEW YORK, NY, 10103-3198, US

(21) Appl. No.: 10/613,359

(22) Filed: Jul. 3, 2003

(51) Int. Cl. G01N 33/53; C12N 1/20

(52) U.S. Cl. 435/252.3

(57) ABSTRACT

A method for screening for antifungal agents is disclosed. This is accomplished via analysis of the activity of certain chaperone proteins, and/or the genes encoding them.

### FIELD OF THE INVENTION

This invention relates to methods for determining agents useful in treating fungal infections, yeast infections in particular. More particularly, it relates to identifying such agents by assaying for their ability to interfere with one or more functions of the CSH3 gene or the CSH3 gene product, or homologs thereof.

### BACKGROUND AND PRIOR ART

Many fungi are implicated in infections of mammals, including humans. Candida albicans is the most common human fungal pathogen. It is implicated in a wide range of superficial mucosal diseases, as well as life threatening systemic infections in immuno compromised subjects. See, e.g., Calderone, et al., Trends Microbiol., 9:327-335 (2001); Carner, et al., Curr. Biol., 7:R691-694 (1997); Calderone, et al., in Calderone, ed., Candida and Candidiasis (ASM Press, Washington, D.C., 2002), pgs. 67-86.

C.albicans, like all microorganisms, must take up nutrients from the environment in order to survive and to proliferate. Apart from this general information, very little is known about the nutrients used by C.albicans during infectious growth within hosts. It is also not well understood if nutrient availability affects the ability of the pathogen to circumvent host protective responses, including phagocytosis by macrophages and neutrophiles. What can be surmised is that, in order to survive within the host, C.albicans cells must be able to adjust the efficacy of nutrient uptake systems rapidly, in response to changes in nutrient levels that are present in microenvironments encountered.

The role of amino acids as metabolites for protein synthesis is well known. They also have an important role in nitrogen homeostasis. It has also been shown that amino acids act as morphogens, with the capacity to induce polymorphic fungi such as C.albicans to undergo morphological transition. See Dabrowski, et al., J. Gen. Microbiol., 127:391-397 (1981); Holmes, et al., J. Gen. Microbiol., 133:3219-3228 (1987). Land et al., Infect. Immun., 11:1014-1023 (1975). Obligate dipolid nature of C.albicans, as well as the lack of any knowledge of a sexual phase, along with unusual codon usage, has hampered study of the organism, including information on amino acid usage. For example, it is not known whether C.albicans assess the availability of amino acids prior to or after being taken up by cells.

Saccharomyces cerevisiae is, of course, a very well studied yeast. It is known to possess very sophisticated systems for importing amino acids from the external environment efficiently. See Forsberg, et al., Curr. Genet., 40:91-109 (2001). Information from studies on S.cerevisiae are relevant because of all the known S.cerevisiae gene products that are involved in positive regulatory circuits that control functional expression of amino acid permeases (“AAPs” hereafter), have C.albicans homologs. These AAPs transport amino acids into cells.

The C.albicans genome is consistent with kinetic studies that indicate multiple transport systems, as 22 ORFs are known which comprise a conserved family of AAPs. Extracellular amino acids present within the growth environment induce expression of several S.cerevisiae AAPs. Induction of these AAPs requires a plasma membrane located sensor complex, the “SPS sensor,” which consists of three parts, i.e., SSY1, PTR3, and SSY5. Cells which lack any member of this complex are unable to respond to amino acid stimuli properly.

The SSY1 protein, or “Ssy1p” is the only integral membrane component, and is the only member of the AAP family that does not transport amino acids. Rather, it is a sensor for external amino acids, and initiates signals that are transduced to peripherally associated plasma membrane proteins, i.e., Ptr3p and Ssy5p. Additionally, Stp1p and Stp2p are redundant transcription factors which bind to specific sequences within the promoters of SPS sensor regulated genes. These proteins are synthesized as latent, cytoplasmic precursors, and are processed, rapidly and endoproteolytically, via induction by SPS, when stimulated by extracellular amino acids. Shorter forms of the proteins, which lack N terminal inhibitory domains, are targeted to the nucleus where they can transactivate SPS sensor target AAP genes.

The members of the AAP family are localized to the plasma membrane and, as other plasma membrane proteins, they are initially cotranslationally inserted into the membrane of the endoplasmic reticulum. Subsequent to this, they attain native conformation prior to transport from the ER to Golgi apparatus, via ER derived transport vesicles.

The members of the yeast S.cerevisiae family of AAPs all require Shr3p to exit the ER. In cells which lack SHR3, the AAPs accumulate in the ER, and Shr3 mutants are unable to respond to external amino acid cues, and have a greatly reduced capacity to take up amino acids.

Diploid strains which carry homozygous Shr3 null mutations undergo dimorphic transitions at enhanced frequencies, and exhibit excessive pseudohyphal growth. Structurally, Shr3p is an integral membrane component of the ER, with 4 membrane spanning segments, and a hydrophobic, cytoplasmically oriented carboxy terminal domain. It physically associates with AAPs, but not with other polytopic membrane proteins. It facilitates membrane association and assembly of soluble vesicle coat forming components. These activities enable Shr3p to prime ER derived transport vesicle formation in the immediate vicinity of AAPs. Thus, Shr3p functions as a packaging chaperone which directs formation of vesicle buds around AAPs, thereby ensuring their inclusion into transport vesicles.

The central role of Shr3p in S.cerevisiae amino acid uptake led to studies on its C.albicans homolog, Csh3p. This work, which is set forth in the experiments which follow, details the role of Csh3p in amino acid uptake by C.albicans, and provides a way to determine if a particular substance has use as an inhibitor of C.albicans. As will be seen, infra it is now shown that amino acids are important sources of nitrogen for C.albicans in situ, during growth in mammalian hosts. The experiments set forth herein describe novel techniques which, inter alia, define a feature of the invention, which is a methodology to determine if a substance impacts amino acids uptake, and thus functions as an antifungal agent.

### DETAILED DESCRIPTION OF PREFERRED EMBODIMENTS

### EXAMPLE 1

In order to proceed with the goals of the project, it was necessary to secure C.albicans strains which exhibited severe and specific defects in amino acid uptake. Such strains would permit assessment of the importance of amino acids for growth and pathogenicity.

As a first step, both chromosomal copies of “CSH3”, which is the C.albicans homolog of the S.cerevisiae packaging chaperone, Shr3p, were cloned using standard methods and three primers, in two amplification reactions. To do this, the first step involved DNA isolated from C.albicans SC5314, and the two primers F1 and R1 (SEQ ID NOS: 1 & 2: cgaaaatgag cacaagctct tcagcccaca cggcgaa (SEQ ID NO: 1, F1), and gtgagtctgc tgcggatcca tacaccaaa (SEQ ID NO: 2, R1). This resulted in amplification of a 2.2 kb fragment, which corresponded to the allele of CSH3 contained in the contig 19-20174 of the known, diploid assembly. The second step involved the two primers F1 and R2 (SEQ ID NO: 3: cgacgagttt acccgggcga ttttcttt). This resulted in a 1.7 kb fragment, which corresponds to the allele of CSH3 in contig 19-1017.

The fragment includes a 1 kb segment, upstream of the ORF, and one 0.5 kb downstream of it. The two primers used introduce Hind III and Bam H1 restriction sites into the product, which is useful for subsequent manipulation.

The alleles can be distinguished via Southern analysis, using restriction endonuclease ClaI.

Once the PCR products were obtained, they were ligated into commercially available plasmid pCR2.1-TA-TOPO, creating pPM18 and pPM19, respectively. The inserts were then sequenced, using standard methods.

When the inserts were sequenced, the ORFs encoding both alleles of CSH3 were found to differ at several nucleotides, but only one of the differences resulted in an amino acid change at position 213, where the amino acid was Gly or Ser.

The insert of pPM18 contained an ORF which was identical to one of the wild type ORFs in the genome databases. This insert was subcloned into plasmid pRS316, which is described by Sikorski, et al., Genetics, 122:19-27 (1989), and had been digested with HindIII and BamHI. The resulting plasmid is referred to as pPM20.

Once pPM20 was created, a KpnI/SacI fragment thereof was cloned into Bluescript KS(+), yielding plasmid pPM38. Following creation of pPM38, it was manipulated to create pPM40. This plasmid, i.e., pPM40, contained a deletion within the CSH3 ORF from position +121, through +542, which was created by digestion with EcoRV (to cut at +121), and PstI (to cut at +542), and combining the restriction product with Klenow blunted pPM38, to yield pPM40. These two plasmids are CSH3 and cshΔ3 respectively, i.e., the one contains the full length ORF, and the other contains the deletion.

KpnI/SacI fragments containing the CSH3 and cshΔ3 alleles were then cloned into KpnI/SacI digested pSMS44, described by Saporitio-Irwin, et al., Mol. Cell Biol., 158:601-613 (1995), to yield plasmids referred to as pPM44 and pPM45. Each of these plasmids contained a 2.1 kb EcoRV/XbaI DNA fragment which contained CAURA3, previously cloned into SmaI/XbaI digested pUC18.

The orientation of the insert of pPM19 was determined, and a HindIII/BamHI fragment was excised and cloned into a HindIII/BamHI digest of pRS316, to produce pPM22. In turn, pPM22 was digested with SmaI, and an SmaI/NaeI fragment containing the yEGFP3 gene was cloned therein. The resulting plasmid, referred to as pPM47, contains yEGFP3 gene, ligated in frame with CSH3. A KpnI/SacI fragment containing this CSH3-GFP product was then subcloned into KpnI/SacI restricted pSM44, creating plasmid pPM57.

These plasmids were used in the experiments which follow.

### EXAMPLE 2

This example describes the development of the strains of C.albicans and S.cerevisiae used in the later examples.

A ura3/ura3 strain of C.albicans, i.e., CA14, described by Fonzi, et al., Genetics, 134:717-728 (1993), was used to construct C.albicans csh3Δ3 mutants. A method taught by DeBacker, et al, Annu. Rev. Microbiol., 54:463-498 (2000), incorporated by reference, was used. This method, involving transformation by spheroblasts, was first used on Pichia pastoris.

The CA14 strain contains two CSH3 alleles, and these were disrupted, sequentially, by a two step gene replacement strategy. First, plasmid pPM45, described supra and carrying the csh3Δ3 allele and the CaURA3 gene was linearized with PfIMI, using well known methods. This restriction endonuclease cuts in the 5′ upstream region of CSH3.

Linearized pPM45 was introduced into the CA14 strain, under conditions favoring homologous recombination. Resulting Ura⁺ transformants were selected. These selectants contained the csh3Δ allele, the URA3 gene, and CSH3 wild type allele, in tandem, on a chromosomal, CSH3 locus.

Any spontaneous Ura− papillants were selected via passage on media containing 5-fluororotic acid. Any papillants which had lost wild type CSH3 but which retained the csh3Δ mutant were identified. One of the resulting, heterozygous mutant strains, PMRCA8, was subjected to a second round of directed integration, loop out mutagenesis, resulting in strain PMRCA10, which is a homozygous, csh3Δ/csh3Δ null mutant strain.

Wild type CA14, PMRCA8, and PMRCA10 were then made Ura3⁺, by integrating a 4.9 kilobase, BglII/PstI fragment which contained the CAURA3 gene into the endogenous URA3 locus, to generate strains PMRCA18, PMRCA19, and PMRCA20, respectively. The CSH3 gene was then introduced back into its locus via transforming PMRCA10 with plasmid pPM44, linearized as described, supra, to obtain PMRCA13. A further strain was produced by transforming PMRCA10 with pPM57, linearized with BspEI. This endonuclease cuts in the promoter region of the CSH3-GFP allele, referred to supra. (Previously, it had been determined that the CSH3-GFP fusion protein was functional, based upon growth assays where the strain grew well on medium where tryptophan was the sole nitrogen source, and was also sensitive to the toxic, lysine analog 2-aminoethyl-L-cysteine).

These strains were then used in the experiments which follow.

### EXAMPLE 3

These experiments describe the culturing of the strains described supra.

Standard culture conditions and media for S.cervesiae and C.albicans have been described by Sherman, in Guthrie, ed. Guide to Yeast Genetics And Molecular Biology (Academic Press, 1991), pp. 3-21, incorporated by reference. To elaborate, Ura⁻ strains of C.albicans were grown in standard media, supplemented with 25 μg/ml of uridine. Tests on S.cerevisiae were carried out in SD medium supplemented with histidine, as well as various toxic amino acid analogs, i.e., L-canaranine (1 μg/ml), L-azetidine-2-carboxylate (500 μg/ml), B-chloro-D, L-alanine (100 μg/ml), p-fluoro-D, L-phenylalanine (400 μg/ml), D, L-ethionine (300 μg/ml), and 2-aminoethyl-L-cysteine (225 μg/ml). Ljungdahl, et al., Cell, 71:463-478 (1992), was followed to produce SPD and SPD containing 30 mM histidine.

Cell suspension of shr3Δ6 S.cervesiae strain FGY145 described by Gilstring, et al., Mol. Cell Biol., 10:3549-3565 (1999), were transformed with either control plasma pRS316, pPL210, which encodes SHR3 and is described by Ljungdahl, et al., supra, or pPM20, which encodes CSH3, and is described supra. The transformants were spotted on the described SD media, and grown in the presence of the toxic amino acids described supra. Suspensions of the cells were also grown on SPD, and SPD containing 30 mM histidine. To determine ability to handle the toxic amino acids, culture plates were incubated, at 30° C. for six days, and photographed.

It had been demonstrated previously that in shr3 mutant strains, AAPs are retained in the endoplasmic reticulum, and as a result of the reduced levels in the PM, the shr3 mutants exhibit poor growth on medium which contains proline as the sole nitrogen source, and are resistant to toxic concentrations of histidine and many toxic amino acid analogues.

Following transformation, all Ura+ transformants grow equally well on ammonium based, minimal media, while those containing the control grow poorly on proline based medium, and were resistant to 30 mM histidine and the toxic amino acids tested.

In contrast, transformants carrying SHR3 grew well on the proline containing medium, but were sensitive to 30 mM histidine and toxic amino acid analogues. The cells transformed with CSH3 were indistinguishable from the SHR3 transformants, in terms of growth pattern.

These experiments show conclusively that CSH3 expression in S.cervesiae fully complements shr3 null mutants, and the CSH3p functions as a packaging chaperone to facilitate exit of AAPs from the endoplasmic reticulum.

### EXAMPLE 4

Following the results in example 3, it was posited that Csh3p had an equally important role in C.albicans. The strains described supra, were developed to test this. To reiterate, wild type Ura⁻ strain C.albicans was used to create a heterozygous, CSH3/csh3Δ mutant, PMCRA8, and a homozygous csh3Δ/csh3Δ mutant, PMCRA10.

It has been known for some time that Ura⁻ strains of C.albicans display a reduced capacity to undergo morphological changes, as compared to Ura⁺ cells. As the consensus is that Ura⁺ phototrophic strains should be used in experiments involving metabolism and development, CaURA3 was reintegrated into one of its loci to produce PMRCA18, which is a Ura⁺, CSH3/CSH3; PMRCA19, which is Ura⁺, CSH3/csh3Δ, and PMRCA12, which is Ura⁺, csh3Δ/csh3Δ. To control for the possibility of transformation induced mutations, PMRCA13 was produced, which is csh3Δ/csh3Δ::CSH3-URA3.

The uptake of amino acids on the C.albicans strains was tested using similar methods as were for S.cerevisiae. The csh3Δ/csh3Δ mutant showed decreased capacity for transport of each amino acid, as compared to CSH3/CSH3. No measurable uptake of Lys or Phe was found in the csh3Δ/csh3Δ mutant, while His and Pro uptake were diminished. The heterozygous strain showed, slight, but significant decreases in the rates of Lys, Phe and Pro uptake as compared to the CSH3/CSH3 strain, suggesting that both copies are expressed in wild type strains. The heterozygous strains did not show decreased capacity to transport adenine. A slight increase in adenine uptake was observed in these strains.

### EXAMPLE 5

These experiments were designed to test the capacity of csh3Δ mutants to affect cell growth on different nitrogen sources. To test this, strains PMRCA18, PMRCA19, PMRCA12, and PMRCA13 were prepared as equally dense suspensions, and were then spotted onto succinate buffered YNB containing ammonium, or 3 mM of allantion, glutamine, glycine, proline, tryptophan, or citrulline as sole nitrogen source. Similarly, aliquots of equally dense suspensions were spotted onto SUD medium (SUD=buffered YNB containing 3 mM urea as sole nitrogen source) containing the lysine analog, 2-aminoethyl-L-cysteine. The cultures were incubated for 3 days at 30° C., and as a control, aliquots were spotted onto YPD, and incubated at 30° C., for 12 hours. Cultures were observed in the same way described supra.

All strains grew equally well when non-amino acid sources of nitrogen were used (ammonium, allatoin, urea). The csh3Δ/csh3Δ mutant grew less well on amino acid containing media than CSH3/CSH3 wild type cells. The contrast was strongest with tryptophan, proline and citrulline, and much more subtle with glutamine and glycine.

These results, which show a pleiotropic effect of the csh3Δ mutant, confirm the importance of Csh3p in facilitating amino acid uptake in C.albicans.

### EXAMPLE 6

These experiments were designed to ascertain the intracellular location of Csh3p.

The construction of CSH3-GFP fusion protein encoding vector pPM47 is described supra. Cells of strain PMRCA15, which was transformed with this vector, were prepared for microscopic evaluation, following growth in YPD. The analysis was carried out via standard methods, including viewing via Nomarski optics, yEGFP fluorescence, and staining with DAPI.

The cells showed bright perinuclear rim staining, which often extended in a filamentous manner into cytoplasm. The pattern of fluorescence for Csh3p was similar to that of Shr3p and other S.cervesiae ER proteins. The localization of Csh3p in the ER is consistent with Csh3p acting analogously to Shr3p as an AAP specific packaging chaperone.

### EXAMPLE 7

These experiments were designed to determine if growth phenotypes, such as that discussed supra, are gene dosage dependent.

Aliquots of suspensions of C.albicans strains containing equal numbers of cells (PMRCA18, which is CSH3/CSH3 (+/+); PMRCA19, which is CSH3/csh3Δ (+/−), PMRCA12, which is csh3Δ/csh3Δ (−/−), and PMRCA13, which is csh3Δ/csh3Δ3::CSH3 (−/−::+), were spotted onto SUD medium with urea as the sole nitrogen source, this medium with toxic lysine derivative 2-aminoethyl-L-cysteine, and buffered YNB containing 3 mM L-citrulline as sole nitrogen source. Culture plates were incubated at 30° C. for 3 days. Aliquots were also spotted onto YPD, and were incubated at 30° C. for 12 hours.

When SUD with urea as the sole nitrogen source was used, CSH3/CSH3 (+/+) strains were sensitive to the 2-aminoethyl L-cysteine, while the csh3Δ (−/−) null mutant was resistant, and exhibited robust growth. Heterozygous strains grew significantly better than wild type, but not as well as the null mutants. This pattern was observed when L-citrulline was used as the sole nitrogen source, and when YPD was the medium. Heterozygous strains grow less well compared to the wild type strain, but better than the homozygous null mutant. In YPD medium, doubling times were 1.2 hours for the (+/+) strain, 1.3 hours for the (+/−), 1.6 hours for the (−/−), and 1.3 hours for the (−/−::+) strain. This indicates that heterozygous strains exhibit amino acid uptake defects with a degree of severity intermediate to the homozygous strains.

Cells were then grown on YNB medium, containing varying concentrations of lysine (0, 0.1, 1 and 3 mM), as sole nitrogen source. A striking gene dosage effect was observed. As expected, none of the strains were able to grow in the absence of lysine, whereas they grew weakly when 0.1 mM lysine was present, showing the C.albicans can metabolize lysine. The ability of the csh3Δ/csh3Δ strain to grow is attributed to either uptake of lysine via residual permeases that are present in greatly reduced amounts, or by non-specific systems. Consistent with this, the growth of the homozygous null mutant improved noticeably when lysine concentration increased. The CSH3/CSH3 (+/+) strain, in contrast, showed no increase in growth rate as lysine concentration increased, and actually showed a decrease at concentrations above 1 mM . Impaired growth of wild type strains is probably due to the inability to restrict lysine uptake, suggesting lysine has a toxic effect when intracellular concentrations are too high. Heterozygous strains exhibited robust growth at concentrations greater than 1 mM . In toto, these results show that C.albicans can use lysine as a nitrogen source, if its import can be slowed sufficiently to mitigate toxic effects. The heterozygous strains have balanced the opposing activities.

### EXAMPLE 8

C.albicans is a polymorphic fungus, which can grow as a budding yeast, or in filamentous form. See Ernst, Contrib. Microbiol., 5:98-111 (2000). The filamentous forms may be hyphae or pseudo-hyphae. In the laboratory, C.albicans cells undergo morphological transitions in response to many environmental stimuli. Switching from yeast to filamentous growth is favored by temperature shifts above 35° C., maintenance of cultures at pH greater than 7, limiting nutrient levels, and the introduction of serum and certain amino acids to the culture medium. In S.cerevisiae, dimorphic transitions resulting in pseudohyphal growth are greatly enhanced in diploid strains which are homozygous for shr3 mutants. See Gimerno, et al., Cell, 68:1077-1090 (1992). The great degree of functional conservation between Csh3p and Shr3p suggested that csh3Δ/csh3Δ C.albicans strains would exhibit altered capacity to undergo morphological transformation. Two possibilities were envisioned. First, if nitrogen limitation which results from the inability to take up amino acids generates an inducing signal (which is the case in S.cerevisiae), then strains which lack CSH3 should exhibit increased propensity to filament. In the alternative, if amino acids provide stimulatory signals, either directly or indirectly, then C.albicans which lack CSH3 would exhibit a reduced ability to filament.

To test this, aliquots of suspensions of equal numbers of PMRCA18, PMRCA19, PMCRA12, and PMRCA13 were spotted on 10% serum agar, and on solid spider medium (10 g/l nutrient broth, 10 g/l mannitol, 2 g/l KHPO₄, 20 g/l agar. See, Liu, et al., Science, 266:1723-1726(1994)). The culture plates were incubated at 37° C. for 5 days, and the resulting grant colonies were examined. Hyphal growth was assessed by examining the edges of these colonies, which form from multiple cells.

The csh3Δ/csh3Δ (−/−) mutants exhibited clearly reduced hypha on both media. In contrast, extensive, indistinguishable levels of hyphal growth were observed surrounding the (+/+), (+/−), and (+/−::+) strains, thus showing the filamentation defects are recessive.

The morphology of grant colonies was also affected by the mutation. The homozygous, (−/−) colonies were smooth, whereas all others were wrinkled.

These experiments show that wild type and heterozygous cells switch morphology in response to amino acids in medium, demonstrating that amino acids act as morphogens.

In follow up experiments, tests were undertaken to determine if strains form hyphae in liquid medium. Aliquots of PMRCA18, PMRCA19, PMRCA12, and PMRCA13 were pregrown in Lee's medium at 25° C. This medium contains several amino acids, biotin, inorganic salts and glucose. It was developed based on the aminopeptidase profile of C.albicans. See, Lee, et al., Sabouradia, 13:148-153 (1975). One characteristic of this medium is that C.albicans exhibits temperature dependent filamentous growth. At 25° C., cells grow in yeast form. When shifted to 37° C., wild type cells rapidly undergo morphological transition and form hyphae.

Aliquots of each strain were grown in Lee's medium at 25° C., and were then used to inoculate fresh medium. The incubation temperature was raised, simultaneously, to 37° C., and the time course of hyphae development was monitored over 24 hours.

At first, all cells exhibited an ovoid shape typical of diploid yeast. At the 1.5 hour point, approximately 60% of cells, regardless of genotype, had small, germ tube-like projections, indicative of responsiveness to the temperature shift. It was not possible to distinguish cultures at this point.

After 5 and 24 hours, however, there were clear differences. At these points, the majority of cells in all but the csh3Δ/csh3Δ (−/−) cultures exhibited long hyphal filaments. These csh3Δ/csh3Δ cells did show slightly more elongated morphology. Presumably, the germ tube-like extensions in the (−/−) cells ceased to elongate, and enlarged to form daughter cells. These elongated cells resembled cph1/cph1 efg1/efg1 double mutant cells, which lack the ability to form hyphae. (See Lo, et al., Cell, 90:939-949 (1997), and rod-like opaque cells (Miller, et al., Cell, 110:292-302 (2002); Sonneborn, et al., Infect. Immun., 67:4655-4660 (1999). Differences in the extent of filamentous growth were apparent when the 24 hour cultures were left stationery for 30 minutes. To elaborate, since they did not filament, the majority of csh3Δ/csh3Δ cells accumulated at the bottom of the tube. The extensive filamentation of cells in other strains resulted in a fluffy mycelial mass, which remained suspended in the medium.

### EXAMPLE 9

Substances other than those described supra, including proline and N-acetyl glucosamine (Glc-Nac) are known to be strong inducers of filamentation in C.albicans. See Holmes, et al, J. Gen. Microbiol., 133:3219-3228 (1987). Further experiments were carried out to ascertain if these substances could, independently induce morphological transitions in (+/+) and (−/−) strains of C.albicans.

To test this, stains were grown in YPD, as described supra, washed, and were resuspended in buffer at an OD₆₀₀ of 1, or buffer (10 MM MES, pH 6.4) containing either 10 mM proline, or 2.5 mM Glc-Nac. The suspensions were incubated for 12 hours, at 37° C., after which the suspensions were examined microscopically.

In the absence of the inducing signal supplied by buffer, neither strain exhibited hyphal characteristics. Wild type cells exposed to proline developed hyphae readily, while only a few cells with short germ tubules were observed in the (−/−) cells. There were no differences observed when Glc-Nac was used, in that both strains showed extensive hyphal development. It can be concluded, therefore, that csh3 null mutants do not affect the capacity of cells to sense and to react to non-amino acid based stimuli.

In further experiments, the null mutants were grown in medium 199, at pH 8, as described by Saporito-Irwin, et al., supra. They consistently formed hyphae, and were indistinguishable from the (+/+) wild type strain.

In follow up experiments, proline uptake was measured over the first 5 hours of incubation by comparing cells in buffer only, and buffer containing 10 mM proline.

The (−/−) cells had low uptake at all times. The uptake capacity of (+/+) cells was unchanged after an hour, but after 5 hours, uptake had dropped to the level of (−/−) cells. In the presence of proline the uptake rate in (+/+) cells dropped significantly faster. It was as low as the (−/−) mutant was after one hour, presumably the result of feedback inhibition.

Taken as a whole, these results indicate that morphological changes are initiated rapidly in response to changing environmental conditions, and not as a result of long term starvation effects. This is consistent with prior work by Dabrowa, et al., J. Gen. Microbiol., 127:391-397 (1981), which showed that a short pulse, of less than an hour, is sufficient to induce germ tube formation.

### EXAMPLE 10

It was of interest to determine the virulence of the various strains.

To do this male BALB/c mice (18-20 g) were injected with one of 0.15 ml saline solution, or 0.15 ml of saline containing 1.2×10⁶ CFUs of PMRCA18, 1.2×10⁶ CFUs of PMRCA19, 1.2 and 1.3×10⁶ CFUs of PMRCA12, or 0.9×10⁶ CFUs of PMRCA13. Ten mice were used for each group. The cells had been grown in YPD, washed, twice, and resuspended in the saline solution to an OD₆₀₀ of 1. Survival of the mice was scored, daily, over a month.

All mice which received PMRCA18 died within 9 days. In contrast, all of the strains which had at least one CSH3 allele deleted showed reduced virulence. With each strain tested, 50% of the mice were alive after 16 days, and with one exception, at least one mouse in each group was alive after 30 days.

Kidneys of two infected mice of each group were removed, and yeast cells isolated, using standard methods. Genotypes of the yeast were analyzed, via PCR, using primers that anneal at positions −938 and +773, relative to the start codon:

| | |
|---|---|
| ggagaatgtg gaccatattc tgca | (SEQ ID NOS: 4 & 5) |
| and | |
| tgagccttat tggttaaccag. | |

PCR was carried out using standard methods.

In all cases, the genotypes of the reisolated strains corresponded to that of the strain that was injected. The data, which are consistent with the haplo insufficiency observed with amino acid uptake and amino acid based growth phenotypes indicate that both copies of CSH3 allele are required for virulence.

The foregoing examples show that amino acids and their uptake by C.albicans have a central role in the growth and virulence of this pathogenic fungus. The protein Csh3p, which is a homolog to the S.cerevisiae protein Shr3p, is shown to have the same critical role in facilitating amino acid uptake in C.albicans as Shr3p does in S.cerevisiae.

With respect to virulence of C.albicans, the data, supra, show that amino acids provide positive morphogenic signals in C.albicans resulting in switching of morphologies. This is important because growth environments within mammalian hosts, such as humans, have high amino acid contents. Interestingly, in vitro experiments showed that while the CSH3/csh3Δ heterozygous strains could properly switch morphologies, this strain exhibited reduced virulence in situ in the mouse model. Therefore, it can be concluded that capacity to switch morphologies does not intrinsically confer advantages in systemic pathogenic infections. The capacity to switch morphologies does not confer pathogenesis intrinsically. The data also show that while C.albicans is prototrophic, it requires high capacity amino acid uptake systems in order to infect mammalian hosts, such as humans, efficiently. C.albicans relies on amino acids within the host to provide the nitrogen required for growth.

The experiments, supra, taken with what is known of S.cerevisiae and Shr3p function, suggest that two distinct processes are affected, negatively, by csh3 mutations. First, AAPs are retained in the ER of the null mutant yeasts, leading to reduced levels of permeases at the PM. Second, C.albicans possesses identifiable homologs of the entire S.cerevisiae SPS sensing pathway suggesting that C.albicans cells access extracellular amino acids. In the shr3Δ null mutant strains the core component of the SPS sensor, Ssy1p, is retained in the ER. Thus, in analogy to what is observed in S.cerevisiae, it can be predicted that the Candida Ssy1p homolog will be retained in the ER. Consequently, since SPS sensor initiated signals are required to depress the transcription of AAP genes, strains carrying homozygous csh3Δ/csh3Δ mutations are expected to exhibit decreased AAP gene expression.

A gene dosage effect was observed for amino acid uptake. There was diminished capacity to use amino acids as sole nitrogen sources, increased resistance to toxic amino acid analogs, and decreased virulence, all of which show that, in heterozygous strains, Csh3p is present in rate limiting amounts. Defects in filamentation, in contrast, are fully recessive. This indicates that morphological transitions are likely to be induced in response to signals initiated from amino acid sensing mechanisms that become amplified by downstream signal transduction pathways. Due to amplification, the molecular switches that engage morphological transition are less sensitive to reduced levels of Csh3p in the heterozygous mutants.

The data generated in the examples is consistent with the possibility that one copy of CSH3 provides cells with sufficient uptake capacity to enable amino acids to accumulate to levels that induce an internal nutrient sensor. The alternate possibility is that the lower levels of Csh3p are sufficient to enable the C.albicans homolog of Ssy1p to reach the PM, thus enabling assembly of functional SPS sensor complexes.

A feature of the invention is methods for determining compounds which are useful in treatment of C.albicans infection, i.e., candidiasis. Mortality from these infections approaches 30%, and strains resistant to current therapies are increasingly prevalent.

The fact that humans and C.albicans are eukaryotic organisms that share many metabolizes pathways renders the fungal pathways regulating the functional expression of AAPs to be of great interest, because human homologs are not known. Further, the reduced virulence of the heterozygous strains increases the attractiveness of Csh3p as a target. Small changes in protein levels result in substantial decreases in fitness of the organism.

Amino acids constitute important virulence factors. All proteins which participate in amino acid uptake, including, but not being limited to, the entire SPS sensor pathway and all AAPs become targets for the development of antifungal agents.

Hence a feature of the invention is a method for screening for agents useful in treating fungal infections, such as C.albicans infections, by contacting a fungal strain with the substance of interest and determining activity of Csh3p, a nucleic acid molecule encoding Csh3p, a homologe of Csh3p, or a nucleic acid molecule encoding the homolog, wherein a change in activity is indicative of a useful therapeutic agent. “Csh3p” activity as used herein, refers to any of the activities referred to herein, including virulence, morphological changes, movement from the ER or lack thereof, levels of the Csh3p protein, levels of Csh3p transcript expression of Csh3p, and so forth. Preferably, the blocking of amino acid uptake is the activity observed.

The invention relates further to screening for possible therapeutic agents for treating other fungal infections. As was note, supra, Csh3p is a homolog of Shr3p in S.cerevisiae showing sequence and structural conservation. Other homologs include Psh3p in Schizosoccharomyces pombe, Aspergillus nidulans, and Botryotinia fuckeliana. Other homologs exist in, e.g., Neurospora crassa. “Infection” as used herein refers to the presence of fungus where it should not be. Exemplary of such infections, but by no means the only types of these, are human infections, infections of domestic or farm animals, fungal infestations of agricultural products, such as stored crops, crops “in the field”, and so forth.

The methodology described herein can be used to identify useful antifungal agents, including small molecules, such as heterocyclic molecules, amino acids and modified forms of the 20-odd standard amino acids, antisense molecules, and so forth. Such agents can be, e.g., inhibitors of function of Csh3p or homologs or inhibitors of expression of their corresponding genes, as well as agents which disrupt the interaction amongst and between proteins which results in exit of amino acid permeases from the ER, or molecules which abolish the ER derived, vesicle priming that is provoked by CSH3.

Yet a further aspect of the invention relates to a method to evaluate virulence, and hence danger posed, by a particular fungal strain. As was shown, supra, amino acids are required for virulence in hosts, and specific morphogenic signals result when the proper amino acids are present. Changes, including switching from yeast to hyphal forms, are measured very easily, as was shown. Hence, culture of a fungus in the presence of nitrogen sources, such as amino acids, can be used to determine virulence and danger.

Yet a further aspect of the invention relates to the ability to determine a possible, anti-fungal agent by determining the effect of a given agent and the specific, and pleiotropic impact of the test compound on uptake of an amino acid. It is well known that compounds are available which essentially poison the fungi by interfering with multitudes of different pathways; however, the work described herein, as well as work described by, e.g., Andréasson, et al., Genes & Development, 16:3158-3172 (December, 2002), incorporated by reference in its entirety, show that genes and their transcription products have specific, pleiotropic effects on amino acid uptake. In addition to the SHR3/CSH3 homologs described supra, materials such as SPS sensor components, including SSY1, PTR3, and SSY5 as well as STP1 and STP2 as discussed by Andréasson, et al., supra, can be monitored once a test compound is added to a culture to determine if it affects the specific amino acid uptake and pleiotropic effect of the gene/transcription product in question.

It is preferred that in the screening, a strain of the fungal organism that has lost one allele for the Csh3p molecule or Csh3p analog be used, but strains which have lost both, or none, may also be used. As with Csh3p, supra, any activity of the homolog may be measured.

Other aspects of the invention will be clear to the skilled artisan an need not be reiterated here.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

1. A method for screening for an agent useful in treating a fungal infection, comprising contacting a fungal sample with a compound to be tested for antifungal activity and determining activity of a Csh3p a Csh3p homolog, a nucleic acid molecule encoding Csh3p or a nucleic acid molecule encoding a Csh3p homolog, as a determination of usefulness of said agent in treating said fungal infection.

2. The method of claim 1, wherein said fungal infection is a yeast infection.

3. The method of claim 1, wherein said fungal infection is a Candida infection.

4. The method of claim 3, wherein said Candida infection is a C.albicans infection.

5. The method of claim 1, wherein activity of Csh3p or Csh3p homolog is virulence.

6. The method of claim 1, wherein activity of Csh3p or Csh3p homolog is hyphal formation.

7. The method of claim 1, wherein said activity is amino acid uptake by fungi in said fungal sample.

8. The method of claim 1, wherein said fungal sample is a sample of fungal cells alleles of which are heterozygous for said Csh3p or Csh3p homolog.

9. The method of claim 1, wherein said fungal sample is a sample of fungal cells which are homozygous recessive for said Csh3p or Csh3p homolog.

10. (Cancelled)

11. The method of claim 1, wherein said infection is a human infection.

12. The method of claim 1, wherein said infection is an infestation of an agricultural crop.

13. A method for determining potential pathogenicity of a fungus, comprising culturing a sample of said fungus in the presence of an amino acid which is found in an environment in which said fungus is found, and determining uptake of said amino acid by said fungus as a determination of potential pathogenicity of said fungus.

14. A method for determining if a compound is an inhibitor of a fungus, comprising contacting said compound to said fungus in the presence of an amino acid taken up via a specific mechanism by said fungus, with a resulting pleiotropic effect and determining if said compound effects said amino acid uptake or pleiotropic effect as a determination of a possible fungal inhibitor.

15. The method of claim 1, wherein said fungal infection is a Schizosaccharomyces infection, an Aspergillus infection, a Botyrotinia infection, or a Neurospora infection.


SEQUENCE LISTING (5 sequences)

SEQ ID NO: 1 - DNA, 37 bp, C.albicans
cgaaaatgagcacaagctcttcagcccacacggcgaa

SEQ ID NO: 2 - DNA, 29 bp, C.albicans
gtgagtctgctgcggatccatacaccaaa

SEQ ID NO: 3 - DNA, 28 bp, C.albicans
cgacgagtttacccgggcgattttcttt

SEQ ID NO: 4 - DNA, 24 bp, C.albicans
ggagaatgtggaccatattctgac

SEQ ID NO: 5 - DNA, 21 bp, C.albicans
tgagccttattggttaaccag